(19) United States
(12) Patent Application Publication
(10) Pub. No.: US 2005/0149048 A1
(43) Pub. Date: Jul. 7, 2005

(54) Surgical instrument of the releaser type for a spinal implant

(75) Inventors: Tiphaine Leport, Lille (FR); Jean-Yves Leroy, Campagne Les Hesdin (FR); Guy Viart, Saint-Leger (FR); Pascal Rokegem, Arras (FR)

Correspondence Address: YOUNG & THOMPSON, 745 SOUTH 23RD STREET, 2ND FLOOR, ARLINGTON, VA, 22202, US

(73) Assignee: EUROSURGICAL SA, Beaurains (FR)

(21) Appl. No.: 10/899,026

(22) Filed: Jul. 27, 2004

(30) Foreign Application Priority Data
Dec. 23, 2003 (FR) 03 15251

(51) Int. Cl. A61B 17/58

(52) U.S. Cl. 606/99

(57) ABSTRACT

A surgical instrument for removal of an immobilizing element includes a main body having a guide tube integral with a fixed handle and having elastic elements for securing the surgical instrument on the head, a locking tube sliding on the guide tube and having elements for immobilizing the surgical instrument on the head, a pusher tube sliding inside the guide tube by way of a mobile handle connected to the fixed handle. The pusher tube has first elements for spacing apart the walls of the head and second elements for immobilizing the connection rod in the base of the head of the bone-anchoring element, and a gripper rod sliding inside the pusher tube and including a gripper device for securing and removal of the immobilizing element from the head of the bone-anchoring element.

The present invention relates to a surgical instrument of the releaser type permitting removal of an immobilizing element from the head of a bone-anchoring element which has been anchored beforehand and belongs to a spinal implant.

The surgical instrument according to the present invention is intended more particularly for spinal implants consisting of a bone-anchoring element which has a U-shaped head intended to cooperate with an immobilizing element which, by way of a clamping screw, permits immobilization, in translation and in rotation, of a connection rod which interconnects the bone-anchoring elements.

The surgical instrument according to the present invention is intended to improve and facilitate the removal of each immobilizing element from the U-shaped head of the corresponding bone-anchoring element.

The surgical instrument according to the present invention consists of:
a main body comprising a guide tube integral with a fixed handle and having elastic means for securing the surgical instrument on the head,
a locking tube sliding on the guide tube and having means for immobilizing the surgical instrument on the head,
a pusher tube sliding inside the guide tube by way of a mobile handle connected to the fixed handle, said pusher tube having first means for spacing apart the walls of the head and second means for immobilizing the connection rod in the base of the head of the bone-anchoring element,
and a gripper rod sliding inside the pusher tube and comprising a gripper device for securing and removal of the immobilizing element from the head of the bone-anchoring element.

The surgical instrument according to the present invention comprises a guide tube which consists, near the handle, of a sleeve having, on its outer periphery, a securing stud supported by an elastic tongue, a free end having a cylindrical internal bore continued by another bore which, in cross section, has a substantially rectangular internal profile matching the external profile of the head of the bone-anchoring element.

The surgical instrument according to the present invention comprises a guide tube which, in the area of its internal bore, has an external profile of rectangular shape delimited by perpendicular walls, such that each first wall has a U-shaped indent intended to cooperate with the connection rod, while each second wall is traversed by a seat opening into the inside of the bore, and in that the guide tube has, on its external profile, flat areas which permit fixation of elastic lamellas integral with a finger which cooperates with the seat and penetrates inside the internal bore.

The surgical instrument according to the present invention has a locking tube which comprises a free end with indents of truncated profile in order to delimit blades intended to exert pressure on the elastic lamellas so as to immobilize the fingers in the head of the implant and, remote from the free end, a gripper handle continued by a cylindrical sleeve which is traversed by a groove intended to cooperate with the stud of the tube permitting guidance of the locking tube during its movement in translation.

The surgical instrument according to the present invention comprises a pusher tube which has an internal bore and a free end having, in the continuation of the bore, another internal bore of substantially rectangular profile matching the external profile of the immobilizing element, and in that the pusher tube comprises, remote from the end, a sleeve having parallel seats of rectangular profile which are intended to receive, respectively, the branches of the fork of the mobile handle, and a groove arranged along the longitudinal axis of the pusher tube and between the seats.

The surgical instrument according to the present invention comprises a pusher tube whose free end is continued, on the one hand, by rectangular blades which are disposed in planes parallel to those containing the walls of the guide tube, and, on the other hand, by fingers which are disposed in planes parallel to those containing the two other walls of the guide tube.

The surgical instrument according to the present invention comprises a pusher tube in which each finger comprises an inclined outer face so that it has a pointed profile.

The surgical instrument according to the present invention comprises a pusher tube whose seats are disposed in a plane parallel to the one containing the inclined fingers which are integral with the free end.

The surgical instrument according to the present invention comprises a gripper rod which, at one of its ends, has a maneuvering handle and, at the opposite end, a gripper device, said gripper rod comprising, between its handle and the gripper device, two sleeves, of which the first sleeve has, on its outer circumference, a stud or pin intended to cooperate with the groove of the sleeve of the pusher tube.

The surgical instrument according to the present invention comprises a gripper rod whose stud or pin is positioned in such a way as to extend along a vertical direction when the maneuvering handle is placed in a horizontal plane.

The surgical instrument according to the present invention comprises a gripper device which, at the end of the gripper rod, is formed by a square or rectangular profile integral, on each of its first parallel faces, with a tongue whose end has a hook-shaped profile, while the other parallel faces of the profile are traversed by a slot opening into an internal bore and continuing in the direction of the second sleeve, and a tumbler which cooperates with the slot and which, when activated, allows the tongues to be spaced apart.

The following description referring to the attached drawings, which are given as non-limiting examples, will permit a better understanding of the invention, of its characteristics, and of the advantages it is likely to afford.

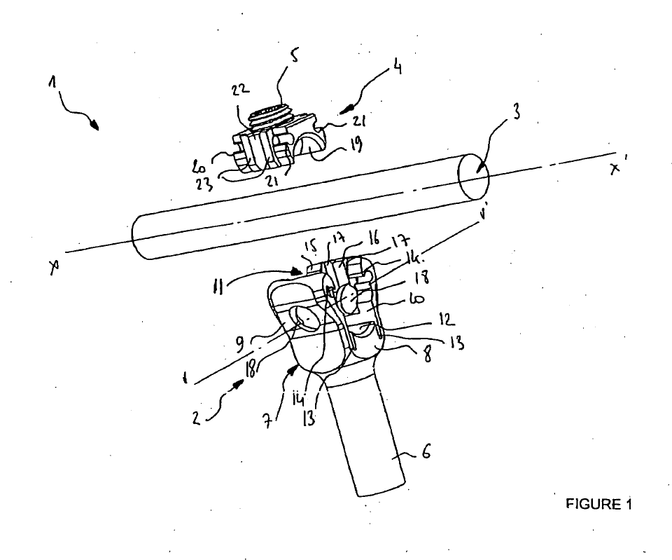

FIG. 1 is an exploded perspective view illustrating, for example, a bone-anchoring element of a spinal implant for which the surgical instrument according to the present invention can be used.

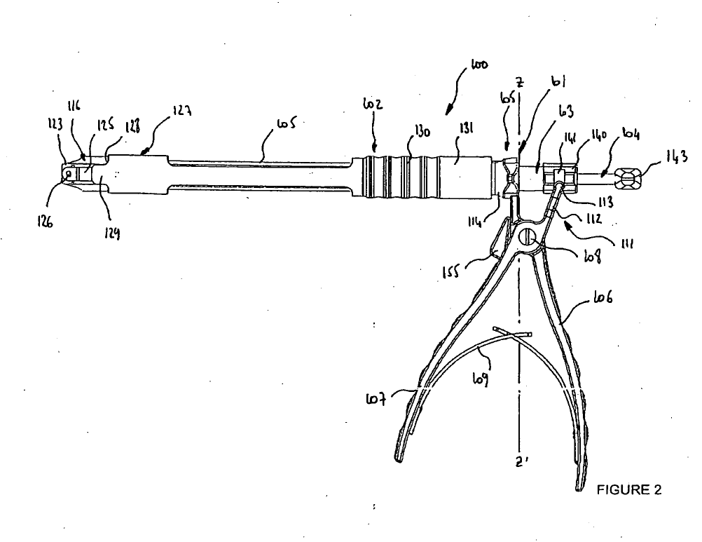

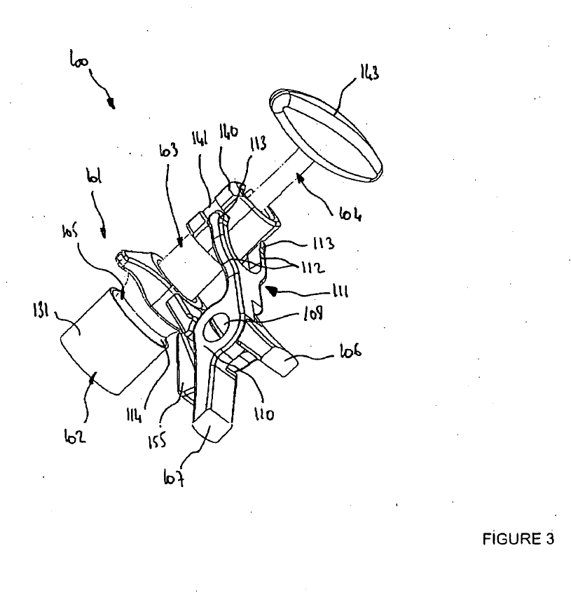

FIGS. 2 and 3 are views showing the surgical instrument according to the invention in the rest position.

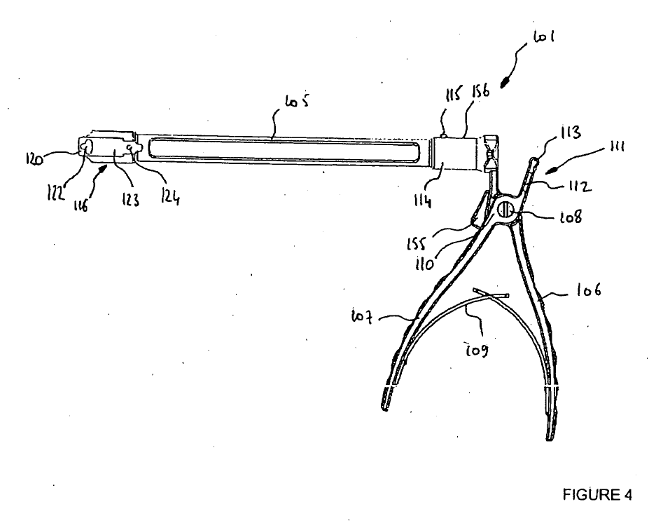

FIG. 4 is a view showing the main body and maneuvering handle of the surgical instrument according to the present invention.

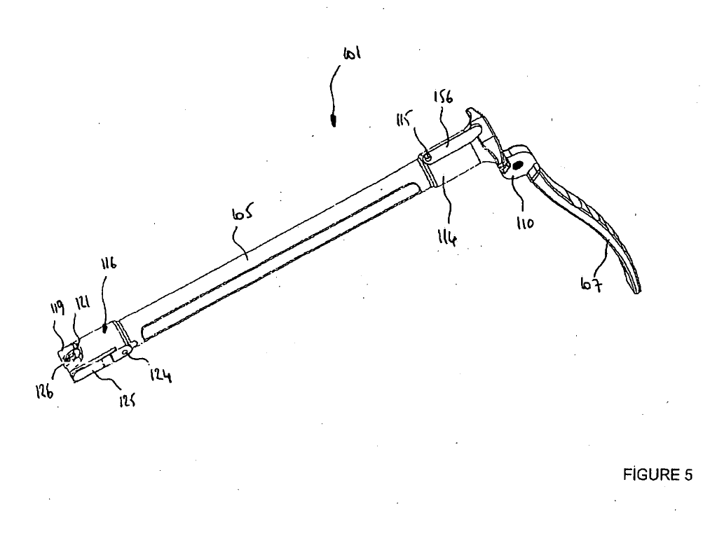

FIG. 5 is a view showing the main body of the surgical instrument according to the present invention.

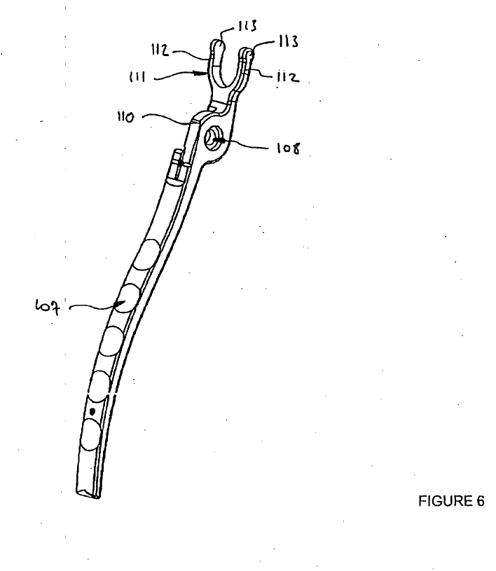

FIG. 6 is a perspective view illustrating the mobile handle of the surgical instrument according to the present invention.

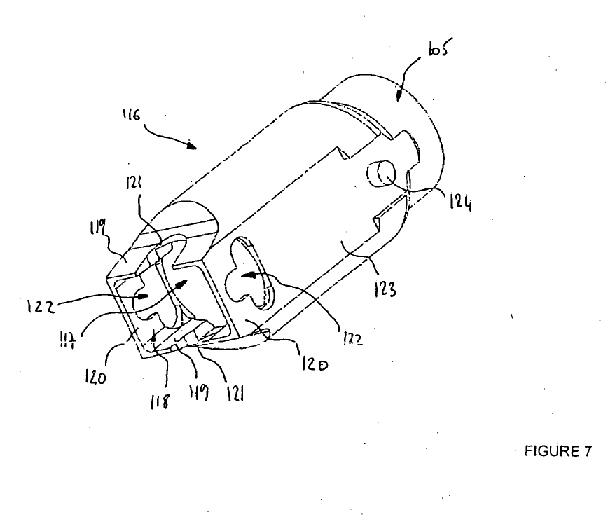

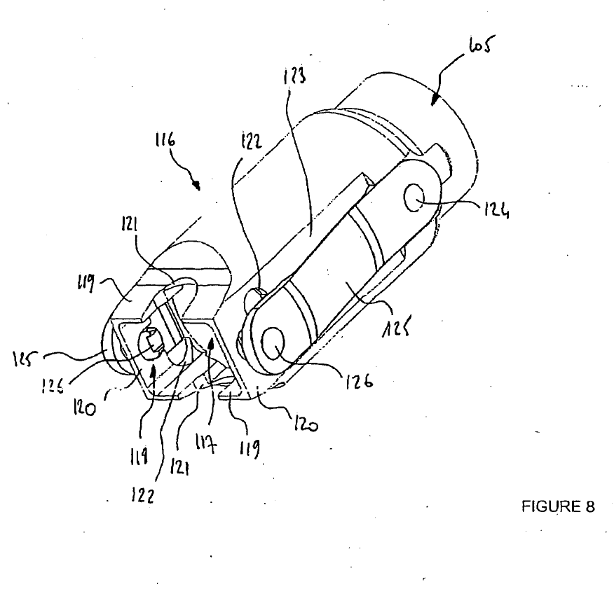

FIGS. 7 and 8 are perspective views showing the main body and more particularly its end remote from the maneuvering handle of the surgical instrument according to the present invention.

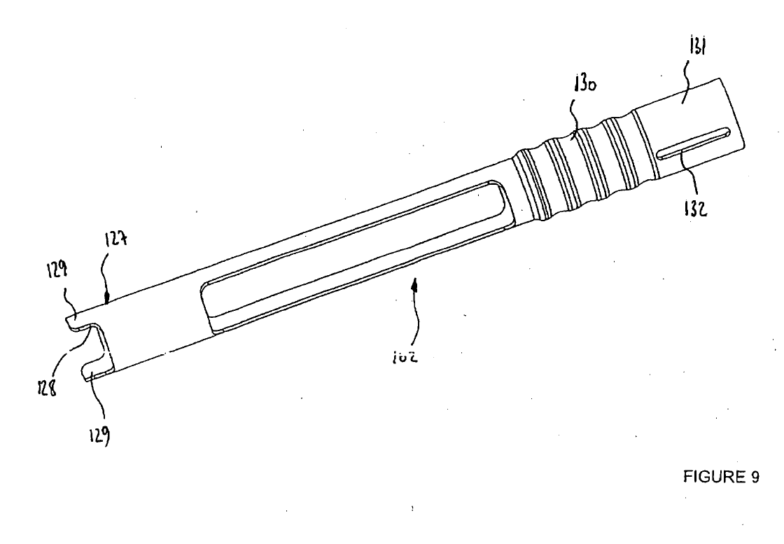

FIG. 9 is a perspective view showing the locking tube of the surgical instrument according to the present invention.

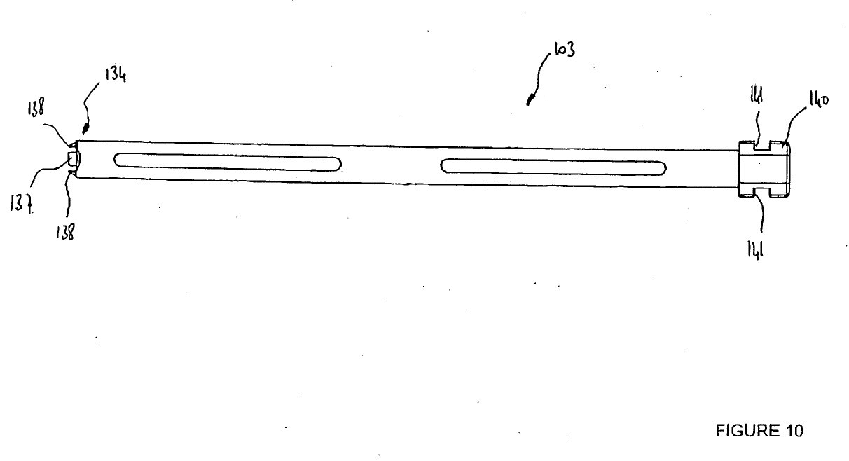

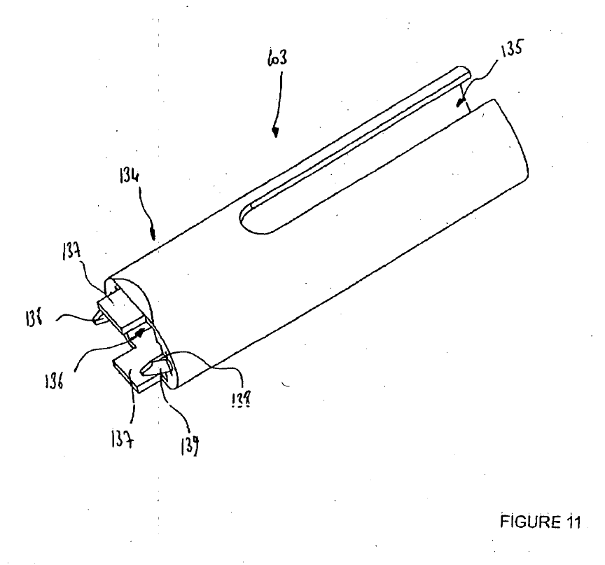

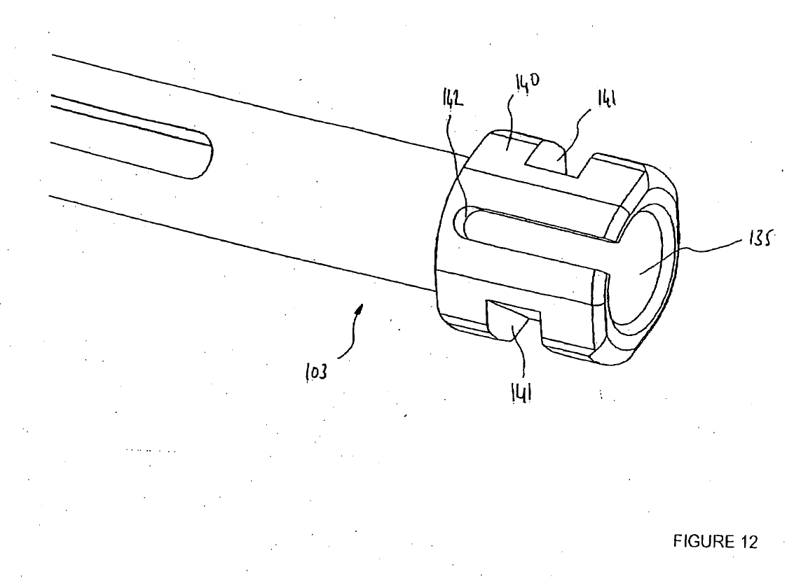

FIGS. 10 to 12 are perspective views illustrating the spacing tube of the surgical instrument according to the present invention.

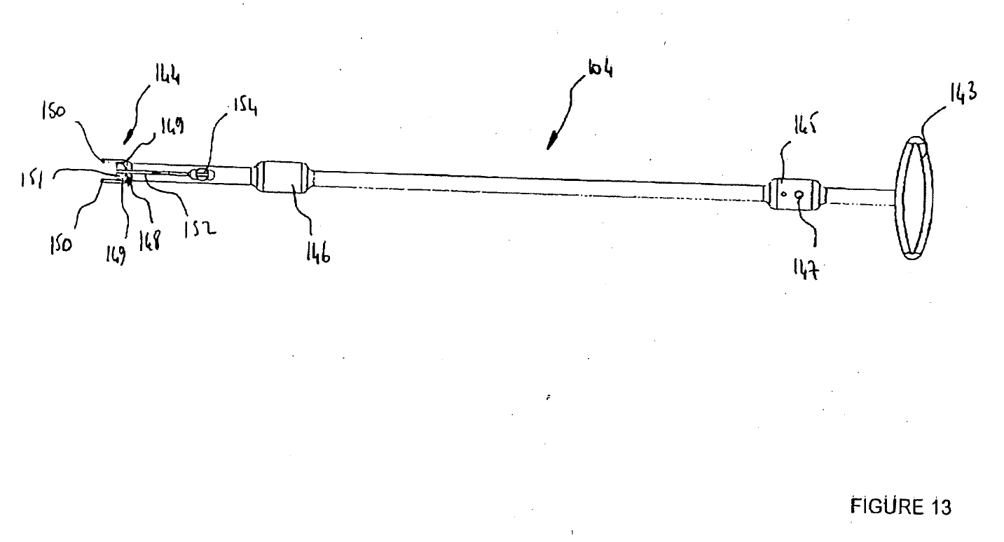

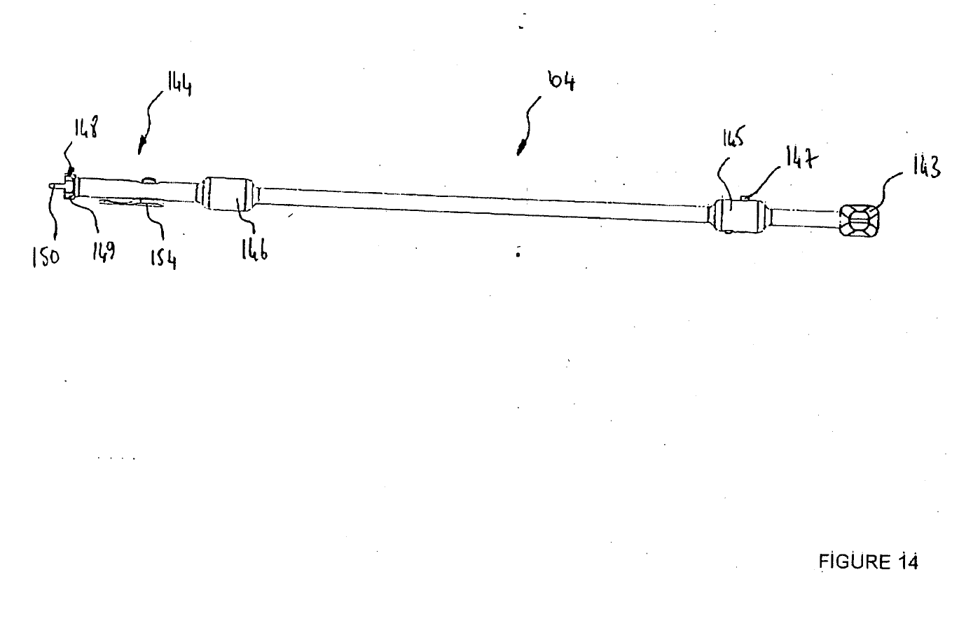

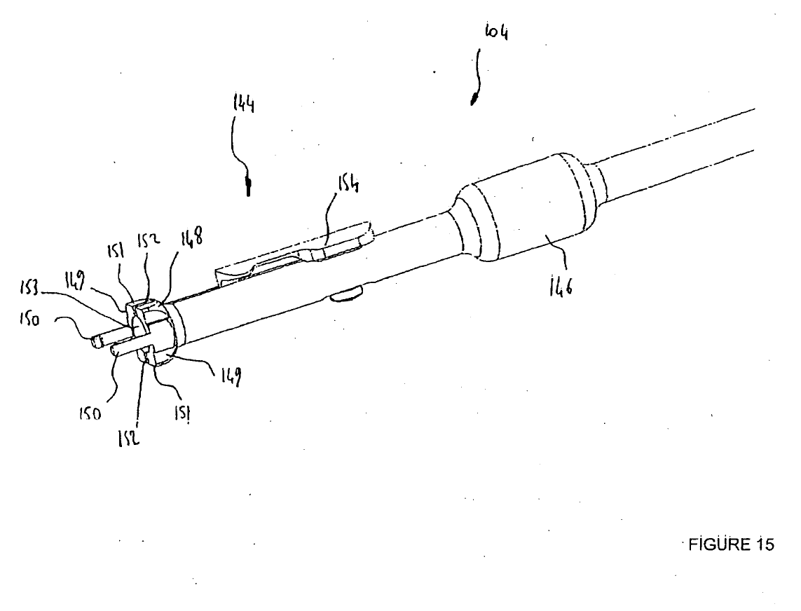

FIGS. 13 to 15 are perspective views showing the gripper rod of the surgical instrument according to the present invention.

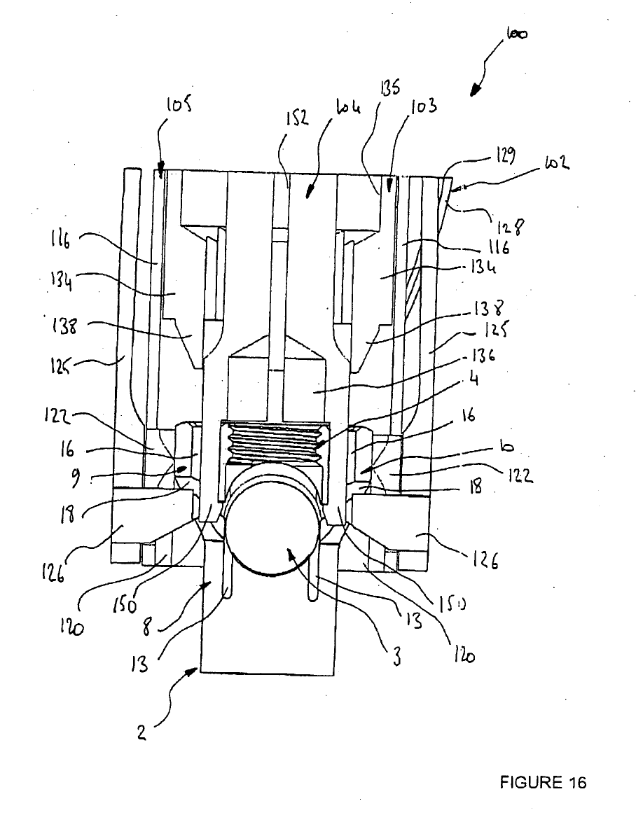

FIGS. 16 to 20 are views showing the different stages in the use of the surgical instrument according to the present invention.

An illustrative embodiment of a spinal implant 1 is shown in FIG. 1, having a bone-anchoring element 2 for immobilizing, in rotation and in translation, a connection rod 3 in the area of each treated vertebra of a vertebral column.

The bone-anchoring element 2 cooperates with an immobilizing element 4 which, by way of a clamping screw 5, allows the connection rod 3 to be fixed in rotation and in translation.

The bone-anchoring element 2 comprises an anchoring part 6 and a receiving part 7 which consists of a U-shaped head 8 open at its upper part so as to cooperate with the connection rod 3 and the immobilizing element 4.

The anchoring part 6 can either be in the form of a hook or have a threaded profile which may or may not be integral with the receiving part 7 so as to be fixed on or in the vertebral body of the vertebra to be treated.

The head 8 comprises two vertical walls 9, 10 disposed opposite one another and in parallel planes in order to delimit a first U-shaped central opening 11 which is arranged on the axis XX′ of the connection rod 3 and whose base 12 has a profile in the shape of a portion of a cylinder.

Each vertical wall 9, 10 is separated, for example, from the base 12 of the central opening 11 by a vertical slit 13 giving a certain elasticity to each wall in the direction of the center of the head 8, along the geometric axis VV′.

The vertical walls 9, 10 respectively comprise, at each end, securing parts 14, 15 disposed opposite one another and on either side of the central opening 11.

Each vertical wall 9, 10 has, on its inner face and between the securing parts 14, 15, a vertical seat 16 with a profile in the shape of a portion of a cylinder, provided on each side with a groove 17 for guiding the immobilizing element 4 when this is fitted in the head 8 of the bone-anchoring element 2.

Between the securing parts 14, 15, each vertical wall 9, 10 is traversed by a hole 18 which opens out inside the central opening 11 and is intended to cooperate with the corresponding surgical instrument.

The immobilizing element 4 has an external profile which is substantially parallelepipedal and which, in a direction parallel to the axis XX′ and in its lower part, has a seat 19 with a profile in the shape of a portion of a cylinder in order to receive and to cooperate with the connection rod 3.

At its center, the immobilizing element 4 has a clamping screw 5 which opens out inside the seat 19 so as to come into contact, with pressure, against the connection rod 3 and immobilize it in rotation and in translation.

In a plane parallel to the axis XX′, the immobilizing element 4 comprises tooth-shaped lugs 20, 21 separated by a vertical seat 22 which is bordered laterally by ribs 23 which guide said immobilizing element during its introduction into the head 8 of the anchoring element 2.

FIGS. 2 to 6 illustrate a surgical instrument 100 permitting removal of the immobilizing element 4 from the U-shaped head 8 of the anchoring element 2. The surgical instrument 100 permits, within the operating site, release of the immobilizing elements 4 from each bone-anchoring element 2 so as to be able to remove the connection rod 3 and/or modify the spinal implant 1.

The surgical instrument 100 is made up of a main body 101, a locking tube 102, a pusher tube 103, and a gripper rod 104.

The main body 101 comprises a horizontal guide tube 105 which, at one of its ends and along a direction parallel to the ZZ′ axis, is integral with a handle 106 referred to as a “fixed handle”.

The fixed handle 106 cooperates with another handle 107 referred to as a “mobile handle” which is connected to the first one via a pivot 108 and a leaf spring 109 making it possible, under the effect of pressure, to move the pusher tube 103 in translation inside the guide tube 105 of the main body 101.

Above the pivot 108, the mobile handle 107 has a head 110 with a fork-shaped profile 111 in which each branch 112 has a free end 113 intended to cooperate with the pusher tube 103 (FIG. 6).

The movement of the mobile handle 107 is limited by a stop element 155 integral with said mobile handle and coming into abutment against the other, fixed handle 106. The stop element 155 can be turned at an angle so as to no longer limit the course of movement of the mobile handle 107.

Near the handle 106, the guide tube 105 consists of a sleeve 114 which, on its outer periphery, has a securing stud 115 intended for guiding the locking tube 102 in translation. The securing stud 115 is mounted on an elastic tongue 156.

FIGS. 7 and 8 show the free end 116 of the guide tube 105 remote from the end integral with the handle 106, and intended to receive the immobilizing element 4 of the spinal implant 1.

The guide tube 105 comprises a cylindrical internal bore 117 which, in the area of its free end 116, is continued by another bore 118 which, in cross section, has a substantially rectangular internal profile matching the external profile of the head 8 of the bone-anchoring element 2 of the spinal implant 1.

In the area of its internal bore 118, the guide tube 105 has an external profile of rectangular shape delimited by perpendicular walls 119, 120.

Thus, each parallel and opposite wall 119 comprises a U-shaped indent 121 intended to cooperate with the connection rod 3 of the spinal implant 1, while each wall 120 is traversed by a T-shaped seat 122 which opens into the inside of the bore 118.

The guide tube 105 has, on its external profile and in a plane perpendicular to the one containing the walls 119 of the internal bore 118, flat faces 123 which continue each wall 120.

Remote from the seat 122, each flat face 123 is integral with a stud 124 permitting fixation of an elastic lamella 125 which has a finger 126 cooperating with the seat 122 and penetrating inside the internal bore 118.

FIG. 9 shows the locking tube 102 fitting around the guide tube 105 and able, by translation, to come to bear against the elastic lamellas 125.

The locking tube 102 has a free end 127 with indents 128 of truncated profile in order to delimit blades 129.

By translation of the locking tube 102, these blades 129 come to press gradually on the lamellas 125 of the guide tube 105.

At the end remote from the free end 127, the locking tube 102 comprises a gripper handle 130 whose external profile is fluted in order to facilitate translation of said tube 102 on the tube 105 by the surgeon.

The gripper handle 130 is continued by a cylindrical sleeve 131 which is traversed by a groove 132 along the longitudinal axis of the tube 102. The groove 132 is intended to cooperate with the stud 115 of the elastic tongue 156 of the tube 105 permitting guidance of the locking tube 102 during its translation.

FIGS. 10 to 12 show the pusher tube 103 which slides inside the bore 117 of the guide tube 105 when a pressure is applied on the handles 106, 107 of the surgical instrument 100.

The pusher tube 103 comprises an internal bore 135 and a free end 134 having, in the continuation of the bore 135, another internal bore 136 of substantially rectangular profile matching the external profile of the immobilizing element 4 of the spinal implant 1.

The free end 134 of the pusher tube 103 is continued by rectangular blades 137 which are disposed in planes parallel to those containing the walls 119 of the guide tube 105.

Similarly, the free end 134 of the pusher tube 103 is continued by fingers 138 which are disposed in planes parallel to those containing the walls 120 of the guide tube 105. Each finger 138 comprises an inclined outer face 139 so that it has a pointed profile.

At that end remote from the end 134, the pusher tube 103 comprises a sleeve 140 with an external diameter greater than that of said tube 103. The sleeve 140 has two parallel seats 141 of rectangular profile which are intended to receive, respectively, the branches 112 of the fork 111 of the mobile handle 107.

The seats 141 are arranged in a plane parallel to that containing the inclined fingers 138 integral with the free end 134 of the pusher tube 103. The sleeve 140 has a groove 142 arranged along the longitudinal axis of the pusher tube 103 and between the seats 141.

FIGS. 13 to 15 show the gripper rod 104 which slides inside the bore 135 of the pusher tube 103 of the surgical instrument 100.

At one of its ends, the gripper rod 104 has a maneuvering handle 143 and, at the opposite end, a gripper device 144.

The gripper rod 104 has, between its handle 143 and the gripper device 144, two sleeves 145, 146 which have an external diameter greater than that of said rod.

On its outer circumference, the first sleeve 145 comprises a stud or pin 147 which cooperates with the groove 142 of the sleeve 140 of the pusher tube 103 upon introduction of the gripper rod 104 into the latter. This arrangement means that the pusher tube 103 and the gripper rod 104 can be joined in rotation during use of the surgical instrument 100.

The stud or pin 147 is positioned in such a way as to extend along a vertical direction when the maneuvering handle 143 is placed in a horizontal plane.

At the end of the rod 104 and in the continuation thereof, the gripper device 144 consists of a square or rectangular profile 148 which, on each of its first parallel faces 149, is integral with a tongue 150 whose end has a hook-shaped profile.

The profile 148 has other parallel faces 151 which are perpendicular to the first faces 149 and which each have a slot 152 opening into an internal bore 153.

Each slot 152 is continued in the direction of the sleeve 146 in order to receive a tumbler 154 which, when activated, makes it possible to space apart the tongues 150 of the gripper device 144.

FIGS. 16 to 20 show the different stages in the use of the surgical instrument 100 for removing in situ, that is to say within the operating site, an immobilizing element 4 of an anchoring element 2, for example with the aim of removing the connection rod 3.

Before placing and immobilizing the surgical instrument 100 on the head 8 of the anchoring element 2, the surgeon proceeds to loosen the clamping screw 5 of the immobilizing element 4 so that the latter can move inside the head 8.

The surgical instrument 100 is positioned on the U-shaped head 8 of the anchoring element 2 in such a way that, on the one hand, the connection rod 3 is lodged inside the indents 121 of the guide tube 105 and, on the other hand, the head 8 of the bone-anchoring element 2 is introduced into the internal bore 118 of said guide tube 105 (FIG. 16).

The positioning of the surgical instrument 100 on the head 8 also makes it possible to introduce the fingers 126 of each elastic lamella 125 of the guide tube 105 inside the holes 18 formed in each of the vertical walls 9, 10 of said head 8 (FIG. 16).

The surgical instrument 100 is fixed on the head 8 of the anchoring element 2 by way of the locking tube 102 which is driven in translation on the guide tube 105 in such a way that the blades 129 are brought progressively against and over the elastic lamellas 125 in order to block the fingers 126 inside each hole 18 of said head 8.

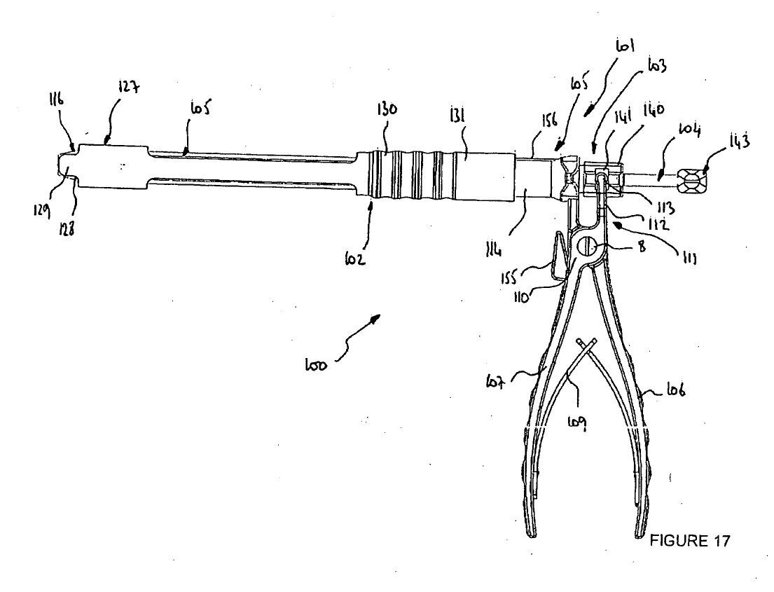

The translation movement of the locking tube 102 on the guide tube 105 is guided by the pin 115 which cooperates with the groove 132 formed in the sleeve 131 of said tube 102 (FIG. 17).

The gripper rod 104 is introduced by the surgeon into the pusher tube 103 until the tongues 150 of the gripper device 144 cooperate respectively with the vertical seat 22 of the immobilizing element 4 and fasten below the latter (FIG. 16).

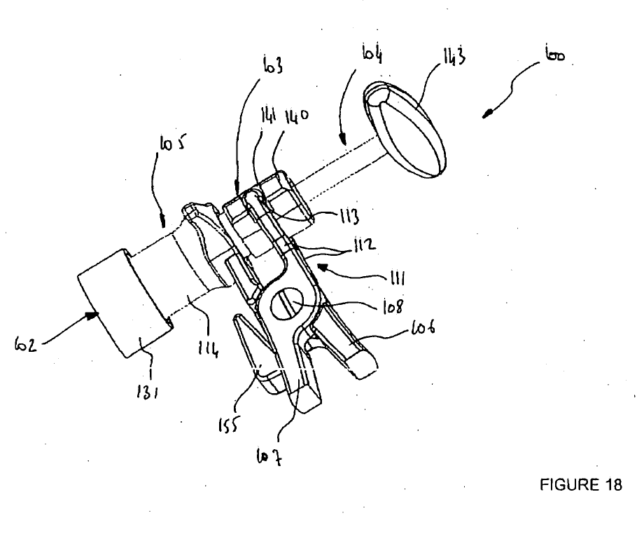

The surgeon actuates the surgical instrument 100 by pressing the mobile handle 107 which, by way of its branches 112, makes it possible to move the pusher tube 103 in translation inside the guide tube 105 and in the direction of the immobilizing element 4 (FIGS. 17, 18).

During this translation movement, the inclined fingers 138 are introduced respectively inside each vertical seat 16 formed in the walls 9, 10 of the head 8 of the bone-anchoring element 2.

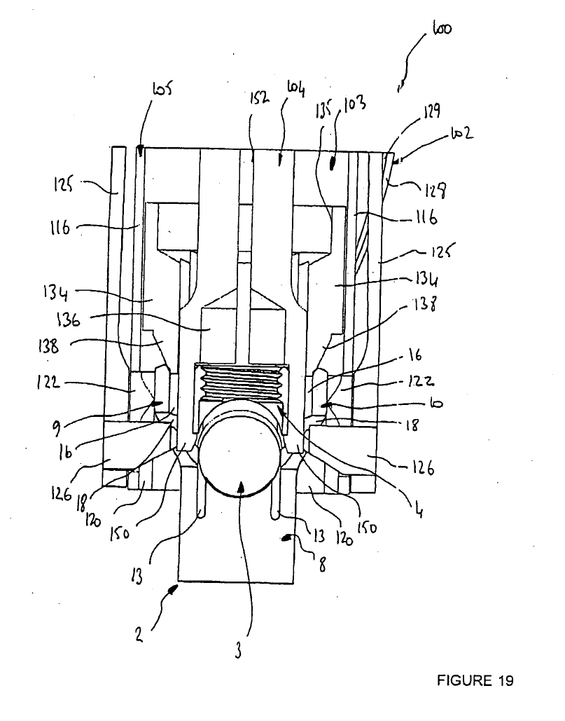

This progressive introduction of the fingers 138 makes it possible to space the walls 9, 10 apart in the outward direction and to free the securing parts 14, 15 of the head 8 from the lugs 20, 21 of the immobilizing element 4 (FIG. 19).

Simultaneously with the introduction of the fingers 138, the rectangular blades 137 come to bear on the connection rod 3 in order to counter any force which it could exert on the immobilizing element 4 at the time of its removal.

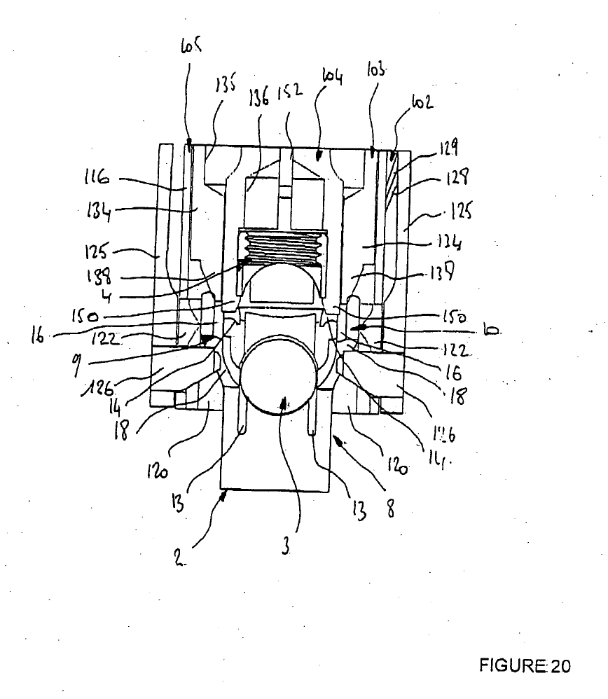

Once the securing parts 14, 15 of the head 8 are spaced apart from the lugs 20, 21 of the immobilizing element 4, the latter is free and can be withdrawn from the bone-anchoring element 2 (FIG. 20).

Thus, the surgeon removes the gripper rod 104 with the aid of the handle 143 of the pusher tube 103 and hence the surgical instrument 100 carrying the immobilizing element 4 retained in the gripper device 144 (FIG. 20).

When the gripper rod 104 is withdrawn from the surgical instrument 100, the surgeon exerts a pressure on the tumbler 154 of the gripper device 144 in order to space apart the slots 152 and release the immobilizing element 4 from the securing tongues 150.

The surgeon can then withdraw the instrument 100 by driving the tube 102 in translation so as to free the elastic lamellas 125 from the pressure exerted by the blades 129. In this way, the elastic lamellas 125 return to their rest position, releasing their fingers 126 from the holes 18 of the head 8 of the bone-anchoring element 2.

Depending on the number of immobilizing elements 4 to be removed, the surgeon can apply the surgical instrument 100 to another head 8 of another anchoring element 2 and proceed with the same steps as have been described above.

The surgeon can then dismantle the surgical instrument 100 by acting on the stop element 155 in order to free the mobile handle 107 and more particularly the branches 112 from the seats 141 of the pusher tube 103.

The release of the mobile handle 107 permits withdrawal of the pusher tube 103 from the guide tube 105.

The locking tube 102 can also be dismantled by simple pressure on the elastic tongue 156, in order to release the pin 115 from the groove 132.

It must also be understood that the above description has been given only by way of example and that it does not in any way limit the invention, and that replacing the described embodiment details with any other equivalents would not constitute a departure from the invention.

1. A surgical instrument for removal of an immobilizing element (4) from the U-shaped head (8) of an anchoring element (2), anchored beforehand in the osseous body of a vertebra, of a spinal implant (1) for fixing a connection rod (3) in rotation and translation, characterized in that it consists of a main body (101) comprising a guide tube (105) integral with a fixed handle (106) and having elastic means (125) for securing the surgical instrument (100) on the head (8), a locking tube (102) sliding on the guide tube (105) and having means (129) for immobilizing the surgical instrument (100) on the head (8), a pusher tube (103) sliding inside the guide tube (105) by way of a mobile handle (107) connected to the fixed handle (106), said pusher tube (103) having first means (138) for spacing apart the walls (9, 10) of the head (8) and second means (137) for immobilizing the connection rod (3) in the base of the head (8) of the bone-anchoring element (2), and a gripper rod (104) sliding inside the pusher tube (103) and comprising a gripper device (144) for securing and removal of the immobilizing element (104) from the head (8) of the bone-anchoring element (2).

2. The surgical instrument as claimed in claim 1, characterized in that the guide tube (105) consists, near the handle (106), of a sleeve (114) having, on its outer periphery, a securing stud (115) supported by a tongue (156), a free end (116) having a cylindrical internal bore (117) continued by another bore (118) which, in cross section, has a substantially rectangular internal profile matching the external profile of the head (8) of the bone-anchoring element (2).

3. The surgical instrument as claimed in claim 2, characterized in that the guide tube (105) has, in the area of its internal bore (118), an external profile of rectangular shape delimited by perpendicular walls (119, 120), such that each wall (119) has a U-shaped indent (121) intended to cooperate with the connection rod (3), while each wall (120) is traversed by a seat (122) opening into the inside of the bore (118), and in that the guide tube (105) has, on its external profile, flat areas (123) which permit fixation of elastic lamellas (125) integral with a finger (126) which cooperates with the seat (122) and penetrates inside the internal bore (118).

4. The surgical instrument as claimed in claim 1, characterized in that the locking tube (102) has a free end (127) with indents (128) of truncated profile in order to delimit blades (129) intended to exert pressure on the elastic lamellas (125) so as to immobilize the fingers (126) in the head of the implant (8) and, remote from the free end (127), a gripper handle (130) continued by a cylindrical sleeve (131) which is traversed by a groove (132) intended to cooperate with the stud (115) of the tube (105) permitting guidance of the locking tube (102) during its movement in translation.

5. The surgical instrument as claimed in claim 1, characterized in that the pusher tube (103) has an internal bore (135) and a free end (134) having, in the continuation of the bore (135), another internal bore (136) of substantially rectangular profile matching the external profile of the immobilizing element (4), and in that the pusher tube (103) comprises, remote from the end (134), a sleeve (140) having two parallel seats (141) of rectangular profile which are intended to receive, respectively, the branches (112) of the fork (111) of the mobile handle (107), and a groove (142) arranged along the longitudinal axis of the pusher tube (103) and between the seats (141).

6. The surgical instrument as claimed in claim 5, characterized in that the free end (134) of the pusher tube (103) is continued, on the one hand, by rectangular blades (137) which are disposed in planes parallel to those containing the walls (119) of the guide tube (105), and, on the other hand, by fingers (138) which are disposed in planes parallel to those containing the two other walls (120) of the guide tube (105).

7. The surgical instrument as claimed in claim 6, characterized in that each finger (138) comprises an inclined outer face (139) so that it has a pointed profile.

8. The surgical instrument as claimed in claim 6, characterized in that the seats (141) are disposed in a plane parallel to the one containing the inclined fingers (138) which are integral with the free end (134) of the pusher tube (103).

9. The surgical instrument as claimed in claim 1, characterized in that the gripper rod (104) has, at one of its ends, a maneuvering handle (143) and, at the opposite end, a gripper device (144), said gripper rod (104) comprising, between its handle (143) and the gripper device (144), two sleeves (145, 146), of which the first sleeve (145) has, on its outer circumference, a stud or pin (147) intended to cooperate with the groove (142) of the sleeve (140) of the pusher tube (103).

10. The surgical instrument as claimed in claim 9, characterized in that the stud or pin (147) is positioned in such a way as to extend along a vertical direction when the maneuvering handle (143) is placed in a horizontal plane.

11. The surgical instrument as claimed in claim 9, characterized in that the gripper device (144) is formed, at the end of the rod (104), by a square or rectangular profile (148) integral, on each of its first parallel faces (149), with a tongue (150) whose end has a hook-shaped profile, while the other parallel faces (151) of the profile (148) are traversed by a slot (152) opening into an internal bore (153) and continuing in the direction of the second sleeve (146), and a tumbler (154) which cooperates with the slot (152) and which, when activated, allows the tongues (150) to be spaced apart.
